(19) United States
(12) Patent Application Publication
(10) Pub. No.: US 2015/0313894 A1
(43) Pub. Date: Nov. 5, 2015

(54) COMBINATION TREATMENT FOR ROSACEA

(71) Applicant: Galderma Laboratories Inc., Fort Worth, TX (US)

(72) Inventors: Michael Graeber, Lawrenceville, NJ (US); Matthew James Leoni, Hampton, NJ (US); Nathalie Wagner, Pegomas (FR)

(21) Appl. No.: 14/797,519

(22) Filed: Jul. 13, 2015

Related U.S. Application Data
(63) Continuation of application No. 14/100,450, filed on Dec. 9, 2013, now abandoned.
Application No. 14/100,450 is a continuation of application No. 13/232,134, filed on Sep. 14, 2011, now abandoned.
(60) Provisional application No. 61/387,260, filed on Sep. 28, 2010.

(51) Int. Cl. A61K 31/498; A61K 9/00; A61K 31/4174

(52) U.S. Cl. CPC A61K 31/498; A61K 31/4174; A61K 9/0014

(57) ABSTRACT

The invention relates to a method of treating erythema and/or telangiectasia associated with rosacea in a patient in need thereof by topically administering an effective amount of a combination of brimonidine or a pharmaceutically acceptable salt thereof and oxymetazoline or a pharmaceutically acceptable salt thereof to the site of erythema and/or telangiectasia on the skin of the patient. The invention further relates to topical compositions including the combination of compounds and a pharmaceutically acceptable carrier.

This application is a continuation of U.S. application Ser. No. 14/100,450, filed Dec. 9, 2013, which is a continuation of U.S. application Ser. No. 13/232,134, filed Sep. 14, 2011, which claims priority from U.S. Provisional Application Ser. No. 61/387,260, filed Sep. 28, 2010, the disclosures of which are incorporated herein by reference.

### BACKGROUND OF THE INVENTION

Many people are affected by inflammatory skin disorders that result in unsightly and painful, and itchy rashes, acne, psoriasis, dermatitis, temporary or persistent dilation of blood vessels in the skin, and acne-like skin eruptions, such as macules, papules, nodules, vesicles or blisters and pustules that may ooze or crust. Inflammatory skin disorders often result in intense psychosocial distress. Rosacea is a common inflammatory skin disorder affecting over 10 million people in the United States. Rosacea generally involves the cheeks, nose, chin, and forehead and the typical age of onset is 30 to 60 years. See e.g., Zuber T. J., Rosacea: Beyond First Blush 32 HOSP. PRACT. 188-189 (1997); THE MERCK MANUAL 813-814 (Keryn A. G. Lane et al. eds. 17^{th} ed. 2001). Many people with early-stage rosacea incorrectly assume that they suffer from adult acne, sun or windburn, or the normal effects of aging.

Rosacea develops gradually starting as frequent blushing and frequent irritation of the facial skin. More advanced rosacea is characterized by a vascular stage where patients display increasingly severe erythema (abnormal redness of the skin) and telangiectasia (visible red lines due to abnormal dilatation of capillary vessels and arterioles). Pimple-like eruptions, which may be solid (called papules or nodules) or puss filled (known as pustules) may develop. Such eruptions often look like acne, but closed and open comedones, frequently referred to as whiteheads or blackheads, and commonly present in acne, are not, usually found in rosacea. Later-stage rosacea is characterized by rhinophyma (enlargement of the nose). If left untreated, rosacea can progress to irreversible disfigurement. Rosacea signs and symptoms are often aggravated by sun exposure, changes or extremes in temperature, wind, and consumption of certain foods, such as spicy foods, caffeine, and alcohol.

The exact pathogenesis of rosacea is unknown, but the pathologic process is well described. For example, erythema associated with rosacea is caused by dilation of the superficial vasculature of the face. Zuber T. J., Rosacea: Beyond First Blush 32 HOSP. PRACT. 188-189 (1997).

There is no known cure for many inflammatory skin disorders like rosacea. Standard treatments include avoidance of triggers such as sun exposure, wind exposure, alcohol consumption, spicy foods, and irritating facial cleansers, lotions, and cosmetics. Antibiotics are the traditional first line of therapy. Long-term treatment (5 to 8 weeks or more) with oral antibiotics such as tetracycline, minocycline, doxycycline or clarithromycin may control skin eruptions. Alternative oral treatments include vitamin A medications, such as isoretinoin and antifungal medications. Unfortunately, such oral medications often cause side effects and many people have limited tolerance. Topical treatments, such as topically applied antibiotics and antifungals or steroids, are available but also have limited effectiveness or the use is restricted due to safety considerations. For example, isoretinoin has serious teratogenic side-effects and female patients of child bearing age must use effective birth control or avoid the therapy. Topical treatments include topically applied metronidazole, topically applied steroids, topically applied azelaic acid, topically applied rentinoic acid or retinaldehyde, and topical vitamin C preparations are available but have limited effectiveness and cannot treat all the signs and symptoms. Intervention, such as the laser elimination of blood vessels, is typically a last resort, but may be prescribed if other treatments are ineffective. In patients with nose hyperplasia, surgical reduction may improve the patient's cosmetic appearance, but does not treat the disease itself. Finally mixed light pulse (photoderm) therapy has only proved somewhat effective for symptoms associated with certain inflammatory skin orders like rosacea in some patients. Thus, there remains a need for topical compositions for treatment of inflammatory skin disorders like rosacea and its symptoms.

In U.S. Pat. No. 7,439,241 brimonidine and its pharmaceutically acceptable salts, especially the tartrate salt, have been found to be effective for use as a topical treatment of redness associated with rosacea. In U.S. Patent Publication No. 2005/0165079, oxymetazoline has also been found to be effective for topically treating erythema resulting from rosacea.

In U.S. Patent Publication No. 2006/0264515, at least one α-adrenergic receptor agonist has been found to be effective for use as a topical treatment of telangiectasia associated with rosacea. Examples of the at least one α-adrenergic receptor agonist include brimonidine and a pharmaceutically acceptable salt thereof and oxymetazoline and a pharmaceutically acceptable salt thereof. There is a need for topical treatments for the symptoms of rosacea, such as erythema and telangiectasia, that work better than currently available therapies.

### SUMMARY OF THE INVENTION

The present inventors have discovered advantageous properties of the combination of brimonidine and oxymetazoline. These advantages include, for example, unexpectedly advantageous pharmacokinetics, increased efficacy, reduced side effects, and/or the ability to use unexpectedly low doses.

In one aspect, the present invention relates to a method for treating erythema associated with rosacea in a patient in need thereof, the method including topically administering an effective amount of a combination of brimonidine or a pharmaceutically acceptable salt thereof and oxymetazoline or a pharmaceutically acceptable salt thereof to the site of erythema on the skin of the patient.

In another aspect, the present invention relates to a method for treating telangiectasia associated with rosacea in a patient in need thereof, the method including topically administering an effective amount of a combination of brimonidine or a pharmaceutically acceptable salt thereof and oxymetazoline or a pharmaceutically acceptable salt thereof to the site of telangiectasia on the skin of the patient. In a preferred embodiment, the pharmaceutically acceptable salt of brimonidine is brimonidine tartrate. In another preferred embodiment, the pharmaceutically acceptable salt of oxymetazoline is oxymetazoline hydrochloride.

The brimonidine or a pharmaceutically acceptable salt thereof is preferably present in a minimum amount of about 0.01% and a maximum amount of about 5% based upon the total weight of the composition. Likewise, the oxymetazoline or a pharmaceutically acceptable salt thereof is preferably present in a minimum amount of about 0.01% and a maximum amount of about 5% based upon the total weight of the composition.

In one embodiment, the active ingredients are only brimonidine or a pharmaceutically acceptable salt thereof and oxymetazoline or a pharmaceutically acceptable salt thereof.

The invention also relates to a topical composition including brimonidine or a pharmaceutically acceptable salt thereof; oxymetazoline or a pharmaceutically acceptable salt thereof; and a pharmaceutically acceptable carrier. The pharmaceutically acceptable carrier is preferably selected from the group consisting of lotions, gels, creams, ointments, pastes, unguents, emulsions, aerosols, sprays, solutions, washes, and shampoos.

### DETAILED DESCRIPTION OF THE INVENTION

In one embodiment, the present invention relates to methods of treating erythema associated with rosacea in a patient in need thereof by topically administering an effective amount of a combination of brimonidine or a pharmaceutically acceptable salt thereof and oxymetazoline or a pharmaceutically acceptable salt thereof to the site of the erythema on the skin of the patient.

The major symptom of rosacea is erythema, i.e., the abnormal redness of the skin. The combination of brimonidine or a pharmaceutically acceptable salt thereof and oxymetazoline or a pharmaceutically acceptable salt thereof has been found to be effective in reducing redness associated with rosacea when applied topically to the site of the erythema on the skin of a patient.

In another embodiment, the present invention relates to methods of treating telangiectasia associated with rosacea in a patient in need thereof by topically administering an effective amount of a combination of brimonidine or a pharmaceutically acceptable salt thereof and oxymetazoline or a pharmaceutically acceptable salt thereof to the site of the telangiectasia on the skin of the patient.

Telangiectasia is a symptom rosacea that causes dilations of the superficial blood vessels, such as arterioles and venules. Telangiectasias are visible small, red, purple or blue surface blood vessels that can be located on the face, upper chest, neck or other parts of the body. Telangiectatic blood vessels can present as swollen blood vessels, spider veins, red dermal patches, purple dermal patches, or blue dermal patches.

Brimonidine, i.e., 5-bromo-6-(2-imidazolidinylideneamino) quinoxaline, is a selective alpha-2 adrenergic receptor agonist. Its structure is shown below.

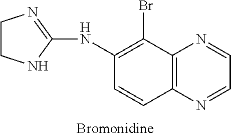 [SMILES: Brc1c(NC2=NCCN2)ccc2nccnc12]

Oxymetazoline is both an alpha-1 and alpha-2 adrenergic receptor agonist. Its structure is shown below.

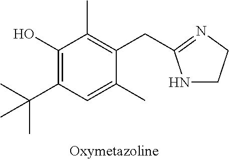 [SMILES: Cc1cc(C(C)(C)C)c(O)c(C)c1CC1=NCCN1]

Pharmaceutically acceptable salts thereof, as used herein, means those salts of the compounds of the invention that are safe and effective for topical use in mammals and that possess the desired biological activity. Pharmaceutically acceptable salts include salts of basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate))salts. Certain compounds of the invention can form pharmaceutically acceptable salts with various amino acids. For a review on pharmaceutically acceptable salts see BERGE ET AL., 66 J. PHARM. SCI. 1-19 (1977).

Brimonidine tartrate is the preferred salt of brimonidine. Oxymetazoline hydrochloride is the preferred salt of oxymetazoline.

The syntheses of brimonidine or a pharmaceutically acceptable salt thereof and oxymetazoline or a pharmaceutically acceptable salt thereof are well known in the art. For example, see U.S. Pat. No. 7,439,241 and Fuhrhop, et al. “Organic Synthesis: Concepts and Methods” 2003, page 237-238.

### Pharmaceutically Acceptable Carriers

In one embodiment, the compounds of the invention are delivered to the affected area of the skin in a pharmaceutically acceptable topical carrier. As used herein, a pharmaceutically acceptable topical carrier is any pharmaceutically acceptable composition that can be applied to the skin surface for topical, dermal, intradermal, or transdermal delivery of a pharmaceutical or medicament. Topical compositions of the invention are prepared by mixing a compound of the invention with a topical carrier according to well-known methods in the art, for example, methods provided by standard reference texts such as, REMINGTON: THE SCIENCE AND PRACTICE OF PHARMACY 1577-1591, 1672-1673, 866-885 (Alfonso R. Gennaro ed. 19th ed. 1995); Ghosh, T. K.; et al. TRANSDERMAL AND TOPICAL DRUG DELIVERY SYSTEMS (1997).

The topical carriers useful for topical delivery of compounds of the invention can be any carrier known in the art for topically administering pharmaceuticals, for example, but not limited to, pharmaceutically acceptable solvents, such as a polyalcohol or water; emulsions (either oil-in-water or water-in-oil emulsions), such as creams or lotions; micro emulsions; gels; ointments; liposomes; powders; aqueous solutions or suspensions, such as standard ophthalmic preparations; aerosols; sprays; washes; and shampoos.

### Emulsions, Gels, Ointments, and Creams as Topical Carriers

In a preferred embodiment, the topical carrier used to deliver a compound of the invention is an emulsion, gel, ointment, or cream. Emulsions, such as creams and lotions are suitable topical compositions for use in the invention. An emulsion is a dispersed system comprising at least two immiscible phases, one phase dispersed in the other as droplets ranging in diameter from 0.1 μm to 100 μm. An emulsifying agent is typically included to improve stability. When water is the dispersed phase and an oil is the dispersion medium, the emulsion is termed a water-in-oil emulsion. When an oil is dispersed as droplets throughout the aqueous phase as droplets, the emulsion is termed an oil-in-water emulsion. Emulsions, such as creams and lotions that can be used as topical carriers and their preparation are disclosed in REMINGTON: THE SCIENCE AND PRACTICE OF PHARMACY 282-291 (Alfonso R. Gennaro ed. 19th ed. 1995).

In one embodiment, the pharmaceutically acceptable carrier is a gel. Gels are semisolid systems that contain suspensions of inorganic particles, usually small inorganic particles, or organic molecules, usually large organic molecules, interpenetrated by a liquid. When the gel mass comprises a network of small discrete inorganic particles, it is classified as a two-phase gel. Single-phase gels consist of organic macromolecules distributed uniformly throughout a liquid such that no apparent boundaries exist between the dispersed macromolecules and the liquid. Suitable gels for use in the invention are known in the art, and may be two-phase or single-phase systems. Some examples of suitable gels are disclosed in REMINGTON: THE SCIENCE AND PRACTICE OF PHARMACY 1517-1518 (Alfonso R. Gennaro ed. 19^{th} ed. 1995). Other suitable gels for use with the invention are disclosed in U.S. Pat. No. 6,387,383 (issued May 14, 2002); U.S. Pat. No. 6,517,847 (issued Feb. 11, 2003); and U.S. Pat. No. 6,468,989 (issued Oct. 22, 2002).

Gelling agents, that may be used include those known to one skilled in the art, such as hydrophilic and hydroalcoholic gelling agents frequently used in the cosmetic and pharmaceutical industries. Preferably, the hydrophilic or hydroalcoholic gelling agent comprises “CARBOPOL®” (B.F. Goodrich, Cleveland, Ohio), “HYPAN®” (Kingston Technologies, Dayton, N.J.), “NATROSOL®” (Aqualon, Wilmington, Del.), “KLUCEL®” (Aqualon, Wilmington, Del.), or “STABILEZE®” (ISP Technologies, Wayne, N.J.).

“CARBOPOL®” is one of numerous cross-linked acrylic acid polymers that are given the general adopted name carbomer. “Carbomer” is the USP designation for various polymeric acids that are dispersible but insoluble in water. When the acid dispersion is neutralized with a base a clear, stable gel is formed. The preferred carbomer is Carbomer 934P because it is physiologically inert and is not a primary irritant or sensitizer. Other carbomers include 910, 940, 941, and 1342.

Carbomers dissolve in water and form a clear or slightly hazy gel upon neutralization with a caustic material such as sodium hydroxide, potassium hydroxide, triethanolamine, or other amine bases. “KLUCEL®.” is a cellulose polymer that is dispersed in water and forms a uniform gel upon complete hydration. Other preferred gelling agents include hydroxyethylcellulose, cellulose gum, MVE/MA decadiene crosspolymer, PVM/MA copolymer, or a combination thereof.

In a preferred embodiment, the minimum amount of gelling agent in the composition is about 0.5%, more preferably, about 0.75%, and most preferably about 1%.

In another preferred embodiment, the maximum amount of gelling agent in the composition is about 2%, more preferably about 1.75%, and most preferably about 1.5%.

In another preferred embodiment, the topical carrier used to deliver a compound of the invention is an ointment. Ointments are oleaginous semisolids that contain little if any water. Preferably, the ointment is hydrocarbon based, such as a wax, petrolatum, or gelled mineral oil. Suitable ointments for use in the invention are well known in the art and are disclosed in REMINGTON: THE SCIENCE AND PRACTICE OF PHARMACY 1585-1591 (Alfonso R. Gennaro ed. 19th ed. 1995).

The pharmaceutical carrier may also be a cream. A cream is an emulsion, i.e., a dispersed system comprising at least two immiscible phases, one phase dispersed in the other as droplets ranging in diameter from 0.1 μm to 100 μm. An emulsifying agent is typically included to improve stability. When water is the dispersed phase and an oil is the dispersion medium, the emulsion is termed a water-in-oil emulsion. When an oil is dispersed as droplets throughout the aqueous phase as droplets, the emulsion is termed an oil-in-water emulsion. Emulsions that can be used as topical carriers and their preparation are disclosed in REMINGTON: THE SCIENCE AND PRACTICE OF PHARMACY 282-291 (Alfonso R. Gennaro ed. 19^{th} ed. 1995).

The pH of the pharmaceutical carrier is adjusted with, for example, a base such as sodium hydroxide or potassium hydroxide. The minimum pH of the carrier is about 5, preferably 5.5, and most preferably 6.2 when the carrier is diluted by a factor of ten. The maximum pH of the carrier is about 7.5, preferably 7, and most preferably 6.8 when the carrier is diluted by a factor of ten. Each minimum pH value can be combined with each maximum pH value to create various pH ranges. For example, the pH may be a minimum of 6.2 and a maximum of 7.5.

The pH values given above are those that occur if the composition is diluted with water by a factor of ten. It is not necessary to dilute the composition by a factor of ten in order to obtain a pH value. In practice, the composition may be diluted by any value that permits pH to be measured. For example, the composition may be diluted by a factor of about five to about twenty.

### Aqueous Topical Compositions of the Invention

In another embodiment, the topical carrier used in the topical compositions of the invention is an aqueous solution or suspension, preferably, an aqueous solution. Well-known ophthalmic solutions and suspensions are suitable topical carriers for use in the invention. Suitable aqueous topical compositions for use in the invention are disclosed in REMINGTON: THE SCIENCE AND PRACTICE OF PHARMACY 1563-1576 (Alfonso R. Gennaro ed. 19th ed. 1995). Other suitable aqueous topical carrier systems are disclosed in U.S. Pat. No. 5,424,078 (issued Jun. 13, 1995); U.S. Pat. No. 5,736,165 (issued Apr. 7, 1998); U.S. Pat. No. 6,194,415 (issued Feb. 27, 2001); U.S. Pat. No. 6,248,741 (issued Jun. 19, 2001); U.S. Pat. No. 6,465,464 (issued Oct. 15, 2002).

Tonicity-adjusting agents can be included in the aqueous topical compositions of the invention. Examples of suitable tonicity-adjusting agents include, but are not limited to, sodium chloride, potassium chloride, mannitol, dextrose, glycerin, and propylene glycol. The amount of the tonicity agent can vary widely depending on the composition's desired properties. In one embodiment, the tonicity-adjusting agent is present in the aqueous topical composition in an amount of from about 0.5 to about 0.9 weight percent of the composition.

Preferably, the aqueous topical compositions of the invention have a viscosity in the range of from about 15 cps to about 25 cps. The viscosity of aqueous solutions of the invention can be adjusted by adding viscosity adjusting agents, for example, but not limited to, polyvinyl alcohol, povidone, hydroxypropyl methyl cellulose, poloxamers, carboxymethyl cellulose, or hydroxyethyl cellulose.

In a preferred embodiment, the aqueous topical composition of the invention is isotonic saline comprising a preservative, such as benzalkonium chloride or chlorine dioxide, a viscosity-adjusting agent, such as polyvinyl alcohol, and a buffer system such as sodium citrate and citric acid.

### Excipients

The topical compositions of the invention can comprise pharmaceutically acceptable excipients such as those listed in REMINGTON: THE SCIENCE AND PRACTICE OF PHARMACY 866-885 (Alfonso R. Gennaro ed. 19th ed. 1995; Ghosh, T. K.; et al. TRANSDERMAL AND TOPICAL DRUG DELIVERY SYSTEMS (1997), including, but not limited to, protectives, adsorbents, demulcents, emollients, preservatives, antioxidants, moisturizers, buffering agents, solubilizing agents, skin-penetration agents, and surfactants.

Suitable protectives and adsorbents include, but are not limited to, dusting powders, zinc stearate, collodion, dimethicone, silicones, zinc carbonate, aloe vera gel and other aloe products, vitamin E oil, allatoin, glycerin, petrolatum, and zinc oxide.

Suitable demulcents include, but are not limited to, benzoin, hydroxypropyl cellulose, hydroxypropyl methylcellulose, and polyvinyl alcohol.

Suitable emollients include, but are not limited to, animal and vegetable fats and oils, myristyl alcohol, alum, and aluminum acetate.

Suitable preservatives include, but are not limited to, quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; alcoholic agents, for example, chlorobutanol, phenylethyl alcohol, and benzyl alcohol; antibacterial esters, for example, esters of parahydroxybenzoic acid; and other anti-microbial agents such as chlorhexidine, chlorocresol, benzoic acid and polymyxin.

Chlorine dioxide (ClO₂), preferably, stabilized chlorine dioxide, is a preferred preservative for use with topical compositions of the invention. The term “stabilized chlorine dioxide” is well known in the industry and by those skilled in the art. Stabilized chlorine dioxide includes one or more chlorine dioxide precursors such as one or more chlorine dioxide-containing complexes and/or one or more chlorite-containing components and/or one or more other entities capable of decomposing or being decomposed in an aqueous medium to form chlorine dioxide. U.S. Pat. No. 5,424,078 (issued Jun. 13, 1995) discloses a form of stabilized chlorine dioxide and a method for producing same, which can be used as a preservative for aqueous ophthalmic solutions and is useful in topical compositions of the invention. The manufacture or production of certain stabilized chlorine dioxide products is described in U.S. Pat. No. 3,278,447. A commercially available stabilized chlorine dioxide which can be utilized in the practice of the present invention is the proprietary stabilized chlorine dioxide of BioCide International, Inc. of Norman, Okla., sold under the trademark Purogene™ or Purite™ Other suitable stabilized chlorine dioxide products include that sold under the trademark DuraKlor by Rio Linda Chemical Company, Inc., and that sold under the trademark Antheium Dioxide by International Dioxide, Inc.

Suitable antioxidants include, but are not limited to, ascorbic acid and its esters, sodium bisulfite, butylated hydroxytoluene, butylated hydroxyanisole, tocopherols, and chelating agents like EDTA and citric acid.

Suitable moisturizers include, but are not limited to, glycerin, sorbitol, polyethylene glycols, urea, and propylene glycol.

Suitable buffering agents for use with the invention include, but are not limited to, acetate buffers, citrate buffers, phosphate buffers, lactic acid buffers, and borate buffers.

Suitable solubilizing agents include, but are not limited to, quaternary ammonium chlorides, cyclodextrins, benzyl benzoate, lecithin, and polysorbates.

Suitable skin-penetration agents include, but are not limited to, ethyl alcohol, isopropyl alcohol, octylphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate); and N-methyl pyrrolidone.

### Additional Pharmaceutical Actives

In one embodiment, the only two pharmaceutically active ingredients in the composition are brimonidine or a pharmaceutically acceptable salt thereof and oxymetazoline or a pharmaceutically acceptable salt thereof.

In another embodiment, one or more additional pharmaceutically active ingredients are included in the composition containing brimonidine or a pharmaceutically acceptable salt thereof and oxymetazoline or a pharmaceutically acceptable salt thereof. Additional active ingredients may include any pharmaceutically active ingredient.

Additional pharmaceutically active ingredients include, but are not limited to, topical corticosteroids and other anti-inflammatory agents, such as betamethasone, diflorasone, amcinonide, fluocinolone, mometasone, hydrocortisone, prednisone, and triamcinolone; local anesthetics and analgesics, such as camphor, menthol, lidocaine, and dibucaine, and pramoxine; antifungals, such as ciclopirox, chloroxylenol, triacetin, sulconazole, nystatin, undecylenic acid, tolnaftate, miconizole, clotrimazole, oxiconazole, griseofulvin, econazole, ketoconozole, and amphotericin B; antibiotics and anti-infectives, such as mupirocin, erythromycin, clindamycin, gentamicin, polymyxin, bacitracin, and silver sulfadiazine; and antiseptics, such as iodine, povidine-iodine, benzalkonium chloride, benzoic acid, chlorhexidine, nitrofurazine, benzoyl peroxide, hydrogen peroxide, hexachlorophene, phenol, resorcinol, and cetylpyridinium chloride.

Use of Topical Compositions of the Invention in Combination with Other Skin-Disorder Treatments

The compositions of the invention can be used alone or in combination with other treatments and medications to provide more effective treatment or prevention of inflammatory skin disorders (e.g., rosacea) and symptoms associated therewith. In a preferred embodiment, the topical compositions of the invention are used in combination with treatment regimens and medications well known for treatment of dermatologic disorders, such as those disclosed in THE MERCK MANUAL 811-830 (Keryn A. G. Lane et al. eds. 17^{th} ed. 2001).

Using a composition or compound of the invention in combination with another medicament or treatment means administering a compound of the invention and the other medicament or treatment to a subject in a sequence and within a time interval such that they can act together to treat or prevent inflammatory skin disorders (e.g., rosacea) and symptoms associated therewith. For example, the compounds of the invention can be administered at the same time as the other medicament in the same or separate compositions or at different times.

Any suitable route of administration can be employed to deliver the additional treatment or medication including, but not limited to, oral, intraoral, rectal, parenteral, topical, epicutaneous, transdermal, subcutaneous, intramuscular, intranasal, sublingual, buccal, intradural, intraocular, intrarespiratory, or nasal inhalation. Thus, the compositions of the invention can be administered together or at separate times with other medications or treatments.

In one embodiment, the topical compositions of the invention are used in combination with systemic administration of antibiotics or retinoids including, but not limited to, orally dosed antibiotics, such as tetracycline, minocin, minocycline, erythromycin, and doxycycline, and orally dosed retinoids such as isotretinoins (e.g., Accutane or Roaccutance).

In another embodiment, the topical compositions of the invention are used in combination with other topical treatments including, but not limited to, topical compositions consisting of metronidizole, hydrogen peroxide, benzoyl peroxide, lipoic acid, and azelaic acid, and sulfur preparations; topically dosed antibiotics, such as metronidazole, clindamycin, and erythromycin; topical retinoids such as tretinoin, adapalene, tazarotene; or topical steroids.

In another embodiment, the topical compositions of the invention are used in combination with mixed light pulse therapy (photoderm), pulsed dye laser treatment, or electrosurgery.

### Dosage

Dosages, dosing frequency, and an effective amount of the compounds of the invention can be determined by a trained medical professional depending on the activity of the compounds of the invention, the characteristics of the particular topical composition, and the identity and severity of the dermatologic disorder being treated.

In general, brimonidine or a pharmaceutically acceptable salt thereof is present in a composition of the invention in a minimum amount of about 0.01%, 0.05%, 0.1%, 0.15%, 0.2%, 0.25%, 0.3%, 0.35%, 0.4%, or 0.5% based upon the total weight of the composition. Generally, brimonidine or a pharmaceutically acceptable salt thereof is present in a composition of the invention in a maximum amount of about 0.5%, 0.6%, 0.7%, 0.8%, 0.9%, 1%, 2%, 3%, 4%, or 5% based upon the total weight of the composition. Particularly preferred dosages of brimonidine or a pharmaceutically acceptable salt thereof are 0.07%, 0.18%, and 0.5%.

In general, oxymetazoline or a pharmaceutically acceptable salt thereof is present in a composition of the invention in a minimum amount of about 0.01%, 0.05%, 0.1%, 0.15%, 0.2%, 0.25%, 0.3%, 0.35%, 0.4%, or 0.5% based upon the total weight of the composition. Preferably, oxymetazoline or a pharmaceutically acceptable salt thereof is present in a composition of the invention in a maximum amount of about 0.5%, 0.6%, 0.7%, 0.8%, 0.9%, 1%, 2%, 3%, 4%, or 5% based upon the total weight of the composition.

It is to be understood that the present invention contemplates embodiments in which each minima is combined with maxima to create all feasible ranges. For example, either (1) brimonidine or a pharmaceutically acceptable salt thereof or (2) oxymetazoline or a pharmaceutically acceptable salt thereof may be present in a composition of the invention in an amount of from about 0.01 percent to about 5 percent based upon the total weight of the composition, preferably, from about 0.1 percent to about 1 percent based upon the total weight of the composition, or more preferably, from about 0.1 percent to about 0.5 percent based upon the total weight of the composition.

In a preferred embodiment, the pharmaceutical composition is delivered topically to the affected area of the skin. To treat the symptoms of rosacea and more specifically erythema and/or telangiectasia, the pharmaceutical compositions of the invention are topically applied directly to the affected area in any conventional manner well known in the art. For example, the compositions are applied by cotton swab or applicator stick, or by simply spreading a composition of the invention onto the affected area with fingers. Generally the amount of a topical composition of the invention applied to the affected skin area ranges from about 0.0001 g/cm² of skin surface area to about 0.01 g/cm², preferably, 0.001 g/cm² to about 0.003 g/cm² of skin surface area. Typically, one to four applications per day are recommended during the term of treatment.

### EXAMPLES

### Example 1

### Gel Composition

| Ingredient | Weight Percent |
|---|---|
| Brimonidine tartrate | 0.18% |
| Oxymetazoline hydrochloride | 0.2% |
| Carbomer 934P | 1.25% |
| Methylparaben | 0.3% |
| Phenoxyethanol | 0.4% |
| Glycerin | 5.5% |
| 10% Titanium dioxide | 0.625% |
| Propylene glycol | 5.5% |
| 10% NaOH Solution | 6.5% |
| DI Water | QS |
| TOTAL | 100% |

### Example 2

### Cream Composition

| Ingredient | Weight Percent |
|---|---|
| Brimonidine tartrate | 0.5% |
| Oxymetazoline hydrochloride | 0.5% |
| Phenoxyethanol | 0.8% |
| Methylparaben | 0.2% |
| Propylparaben | 0.05% |
| Disodium EDTA | 0.01% |
| Butylated Hydroxytoluene | 0.05% |
| PEG-300 | 4.0% |
| PEG-6 Stearate (and) Glycol | 7.5% |
| Stearate (and) PEG-32 Stearate | |
| Cetostearyl alcohol | 4.0% |
| Caprylic capric triglycerides | 7.0% |
| Diisopropyl adipate | 7.0% |
| Oleyl alcohol | 7.0% |
| Lanolin USP | 2.0% |
| Ceteareth-6 (and) Stearyl | 2.0% |
| Alcohol | |
| Ceteareth-25 | 2.0% |
| Tartaric Acid | 0.001% |
| DI Water | 55.389% |
| TOTAL | 100% |

### Example 3

### Ointment Composition

| Ingredient | Weight Percent |
|---|---|
| Brimonidine tartrate | 5.0% |
| Oxymetazoline hydrochloride | 5.0% |
| Cholesterol | 3.0% |
| Stearyl Alcohol | 3.0% |
| White Wax | 8.0% |
| White Petroleum | 76.0% |
| TOTAL | 100% |

### Example 4

### Aqueous Solution

An aqueous solution of the invention includes brimonidine tartrate (0.07 wt %); oxymetazoline hydrochloride (0.07 wt %); Purite® (0.005%) (stabilized chlorine dioxide) as a preservative; and the inactive ingredients: boric acid; calcium chloride; magnesium chloride; potassium chloride; purified water; sodium borate; sodium carboxymethylcellulose; sodium chloride; with hydrochloric acid and/or sodium hydroxide to adjust the pH to 5.6 to 6.6. The osmolality is in the range of 250-350 mOsmol/kg.

Thus, while there have been described what are presently believed to be preferred embodiments of the invention, those skilled in the art will realize that changes and modifications may be made thereto without departing from the spirit of the invention, and it is intended to claim all such changes and modifications as fall within the true scope of the invention.

We claim:

1. A method for treating erythema associated with rosacea in a patient in need thereof, the method comprising topically administering an effective amount of a combination of brimonidine or a pharmaceutically acceptable salt thereof and oxymetazoline or a pharmaceutically acceptable salt thereof to the site of erythema on the skin of the patient.

2. A method according to claim 1, wherein the pharmaceutically acceptable salt of brimonidine is brimonidine tartrate.

3. A method according to claim 1, wherein the pharmaceutically acceptable salt of oxymetazoline is oxymetazoline hydrochloride.

4. A method according to claim 1, wherein the brimonidine or a pharmaceutically acceptable salt thereof is present in a minimum amount of about 0.01% and a maximum amount of about 5% based upon the total weight of the composition.

5. A method according to claim 1, wherein the oxymetazoline or a pharmaceutically acceptable salt thereof is present in a minimum amount of about 0.01% and a maximum amount of about 5% based upon the total weight of the composition.

6. A method according to claim 1, wherein the active ingredients are only brimonidine or a pharmaceutically acceptable salt thereof and oxymetazoline or a pharmaceutically acceptable salt thereof.

7. A method for treating telangiectasia associated with rosacea in a patient in need thereof, the method comprising topically administering an effective amount of a combination of brimonidine or a pharmaceutically acceptable salt thereof and oxymetazoline or a pharmaceutically acceptable salt thereof to the site of the telangiectasia on the skin of the patient.

8. A method according to claim 7, wherein the pharmaceutically acceptable salt of brimonidine is brimonidine tartrate.

9. A method according to claim 7, wherein the pharmaceutically acceptable salt of oxymetazoline is oxymetazoline hydrochloride.

10. A method according to claim 7, wherein the brimonidine or a pharmaceutically acceptable salt thereof is present in a minimum amount of about 0.01% and a maximum amount of about 5% based upon the total weight of the composition.

11. A method according to claim 7, wherein the oxymetazoline or a pharmaceutically acceptable salt thereof is present in a minimum amount of about 0.01% and a maximum amount of about 5% based upon the total weight of the composition.

12. A method according to claim 7, wherein the active ingredients are only brimonidine or a pharmaceutically acceptable salt thereof and oxymetazoline or a pharmaceutically acceptable salt thereof.

13. A topical composition comprising brimonidine or a pharmaceutically acceptable salt thereof; oxymetazoline or a pharmaceutically acceptable salt thereof; and a pharmaceutically acceptable carrier.

14. A topical composition according to claim 13, wherein the pharmaceutically acceptable carrier is selected from the group consisting of lotions, gels, creams, ointments, pastes, unguents, emulsions, aerosols, sprays, solutions, washes, and shampoos.

15. A topical composition according to claim 13, wherein the active ingredients are only brimonidine or a pharmaceutically acceptable salt thereof and oxymetazoline or a pharmaceutically acceptable salt thereof.

16. A topical composition according to claim 13, wherein the brimonidine or a pharmaceutically acceptable salt thereof is present in a minimum amount of about 0.01% and a maximum amount of about 5% based upon the total weight of the composition.

17. A topical composition according to claim 13, wherein the oxymetazoline or a pharmaceutically acceptable salt thereof is present in a minimum amount of about 0.01% and a maximum amount of about 5% based upon the total weight of the composition.
